(19) United States
(12) Patent Application Publication
(10) Pub. No.: US 2004/0267322 A1
(43) Pub. Date: Dec. 30, 2004

(54) Portable defibrillator with bypass line power emergency charging of capacitor

(75) Inventors: Gregory T. Kavounas, Kirkland, WA (US); Richard C. Nova, Kirkland, WA (US); Joseph Bradley Williamson, Mercer Island, WA (US); Stephen B. Johnson, Clinton, WA (US); Daniel Yerkovich, Seattle, WA (US); Patrick F. Kelly, Edmonds, WA (US); Paul S. Tamura, Seattle, WA (US)

Correspondence Address: INTELLECTUAL PROPERTY GROUP, FREDRIKSON & BYRON, P.A., 200 SOUTH SIXTH STREET, SUITE 4000, MINNEAPOLIS, MN, 55402, US

(73) Assignee: Medtronic Physio-Control Corp., Redmond, WA (US)

(21) Appl. No.: 10/608,239

(22) Filed: Jun. 27, 2003

(51) Int. Cl. A61N 1/39

(52) U.S. Cl. 607/5

(57) ABSTRACT

The present invention provides a portable defibrillator having a capacitor adapted to receive an electrical charge to deliver a defibrillation charge. Power terminals are provided to receive line power. A charging circuit is provided to charge the capacitor from line power after the power terminals receive line power. Therefore, the defibrillator is capable of receiving line power, such as standard 120 VAC, to charge the defibrillator's capacitor. By charging the capacitor directly through line power, the capacitor is charged in much less time than searching for and replacing a defibrillator battery.

### TECHNICAL FIELD

The invention relates to medical devices, and in particular, emergency charging capacitors of portable defibrillators.

### BACKGROUND

Each day thousands of Americans are victims of cardiac emergencies. Cardiac emergencies typically strike without warning, oftentimes striking people with no history of heart disease. The most common cardiac emergency is sudden cardiac arrest (“SCA”). It is estimated more than 1000 people per day are victims of SCA in the United States alone.

SCA occurs when the heart stops pumping blood. Usually SCA is due to abnormal electrical activity in the heart, resulting in an abnormal rhythm (arrhythmia). One such abnormal rhythm, ventricular fibrillation (VF), is caused by abnormal and very fast electrical activity in the heart. During VF the heart cannot pump blood effectively. Because blood may no longer be pumping effectively during VF, the chances of surviving decrease with time after the onset of the emergency. Brain damage can occur after the brain is deprived of oxygen for four to six minutes.

Applying an electric shock to the patient's heart through the use of a defibrillator treats VF. The shock clears the heart of the abnormal electrical activity (in a process called “defibrillation”) by depolarizing a critical mass of myocardial cells to allow spontaneous organized myocardial depolarization to resume.

Cardiac arrest is a life-threatening medical condition that may be treated with external defibrillation. External defibrillation includes applying electrodes to the patient's chest and delivering an electric shock to the patient to depolarize the patient's heart and restore normal sinus rhythm. The chance a patient's heart can be successfully defibrillated increases significantly if a defibrillation pulse is applied quickly.

Until recently, only individuals such as paramedics, emergency medical technicians, police officers, and others trained in defibrillation techniques used defibrillators. However, in a cardiac arrest event the patient's need is urgent and the patient cannot wait for trained personnel to arrive. In recognition of the need for prompt treatment, automated external defibrillators (AEDs) are becoming more commonplace, and are available in venues such as health clubs, auditoriums, and most recently private homes. Ready availability of AEDs may mean patients can get needed treatment promptly, and need not wait for emergency personnel to arrive. As a result, more lives may be saved.

An AED may be used infrequently, whether it is placed in a commercial setting or in a private household. The battery within the AED will gradually discharge because of self-discharge and automated self-testing that is conducted on a periodic basis (daily, weekly, etc.). Since the AED is used infrequently, the battery status may not be checked on a regular basis. As a result, when the AED is brought into use, possibly years after purchase, the battery may not have sufficient energy to allow the AED to perform its intended function (ECG analysis and defibrillation).

As part of ordinary maintenance procedures, AEDs deployed may be periodically checked. Typically in public venues a person, such as a security worker, may be assigned to make an inspection of each AED and confirm the device is operational. The inspection may be relatively simple, because many AEDs perform one or more automatic self-diagnostic routines and provide one or more status indications that the device is operational or in need of service.

As part of the inspection, the responsible person regularly reviews each AED and checks its associated status indicators. The responsible person may also be required to prepare and maintain records showing the inspections have been performed, as well as log the status and repair history of the AEDs. However, in a public venue having several AEDs, the cost of inspection may be significant. Further, a deployed AED may be unprepared to provide defibrillation therapy if the responsible person fails to make an inspection, forgets to make an inspection, or makes an inspection error.

These problems are exacerbated in a private venue or a household where an AED may be used even more infrequently, and thus the AED may have a larger chance of not being properly inspected. It may be more likely in a private venue or a household the user will forget about the AED due to the long time periods between AED uses. Thus, there is a greater chance in these private settings the AED battery will not be properly charged to adequately provide a defibrillation pulse.

With reference to FIG. 1, a perspective view of a prior art portable defibrillator and a battery pack are shown. Power for the defibrillator is provided by a battery pack 28 that fits inside a battery well 30 located on the side of the defibrillator. Battery pack 28 is generally shaped like a shoebox. The front 32 of battery pack 28 is formed with a latch 34 that extends across the majority of front 32. Latch 34 is grasped by a user to remove battery pack 28 from the defibrillator. Latch 34 automatically secures battery pack 28 in battery well 30 when battery pack 28 is inserted into the defibrillator. The front 32 of battery pack 28 is formed so when battery pack 28 is inserted into the defibrillator, front 32 of battery pack 28 is flush with the case 22 of the defibrillator.

However, this prior art design has some limitations especially when this defibrillator is utilized in private venues including homes. Defibrillators are not used frequently in private households. Battery pack 28 within the defibrillator will gradually discharge because of self-discharge and automated self-testing conducted on a periodic basis. Since the defibrillator is used infrequently, when the defibrillator is brought into use, possibly years after purchase, battery pack 28 may not have sufficient energy to allow the defibrillator to perform its intended function. The user can purchase an alternate battery pack to exchange with battery pack 28, however, even if an alternate has been purchased and is stored closely to the defibrillator for easy access, the alternate has an equal chance of being depleted due to lack of maintenance. Therefore, it is desirable to provide an emergency power source to allow the defibrillator to perform its intended functions when needed.

With reference to FIG. 2a, a schematic depicting the general circuitry of a prior art defibrillator is shown. Generally defibrillation circuitry 41 is comprised of a non-rechargeable battery 40, which provides a defibrillator charge to capacitor 42 through capacitor charging circuit 44. Capacitor 42 provides a defibrillation pulse to a patient through discharge circuit 46 and electrodes 48. In this prior art circuitry design, when battery 40 is dissipated to a point beyond providing a defibrillation charge as shown in FIG. 2b, then battery 40 must be removed and replaced with an alternate battery, if one is available. This battery replacement can take a relatively large amount of time, particularly for a patient experiencing fibrillation. Even if battery 40 were rechargeable, the recharge process takes a long time typically on the order of several hours to days. Therefore, these power sources are not realistic alternates in the event of a failed battery 40 during an emergency.

Generally, disposable batteries power AEDs. There are presently AEDs, which have an option for using rechargeable batteries. In these AEDs, the batteries must be removed from the AED unit and connected to an AC-powered charger to charge the batteries.

As an alternative to purchasing an alternative battery, there are currently defibrillators, typically called “defibrillator monitors”, offered which allow for the battery to be charged directly from line power while the battery remains inside the defibrillator. However, while these defibrillators charge relatively quickly, they have to be turned on by an operator before the capacitor can be charged. Therefore, in the event of a cardiac event, the defibrillator operator would have to manually turn the defibrillator on and then choose how the capacitor would be charged. Further, there are federal regulations and standards, which require proper insulation between the line power and cardiac victim thus adding to the cost of the defibrillator.

What is needed, therefore, is a defibrillator capable of using line power to charge the defibrillator's capacitor. Presently, line power has only been utilized to charge the defibrillator battery. However, this is slow and cannot be utilized during an emergency.

### SUMMARY

A preferred embodiment of the invention overcomes the problems of the prior art. The invention provides a portable defibrillator, such as an AED, having a capacitor adapted to receive an electrical charge from a main battery and to deliver a defibrillation charge via a regular current path. In addition, power terminals are provided to receive line power. An emergency charging circuit is provided to charge the capacitor from the power terminals via an emergency current path, which is distinct from the regular current path.

An advantage of the present invention is by charging the capacitor directly through line power the capacitor is charged in much less time than searching for and replacing a defibrillator battery. An additional advantage is that line power is available in many locations such as standard wall socket.

### BRIEF DESCRIPTION OF DRAWINGS

FIG. 1 is a perspective view of a prior art portable defibrillator and a battery pack;

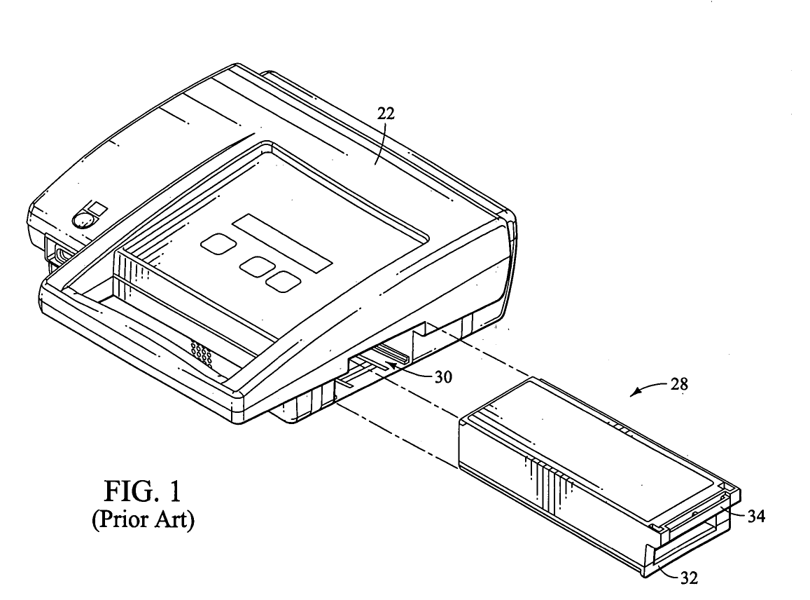

FIG. 2a is a schematic of prior art defibrillation circuitry;

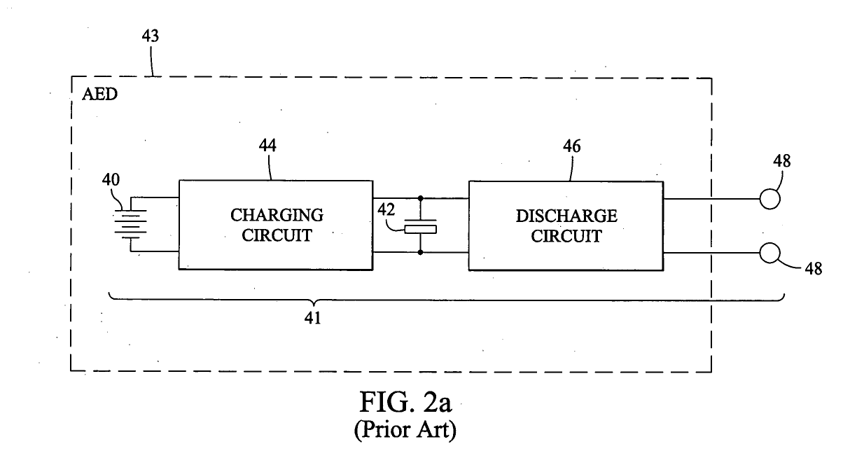

FIG. 2b is a schematic of prior art defibrillation circuitry where the defibrillator has sat for a long period of time;

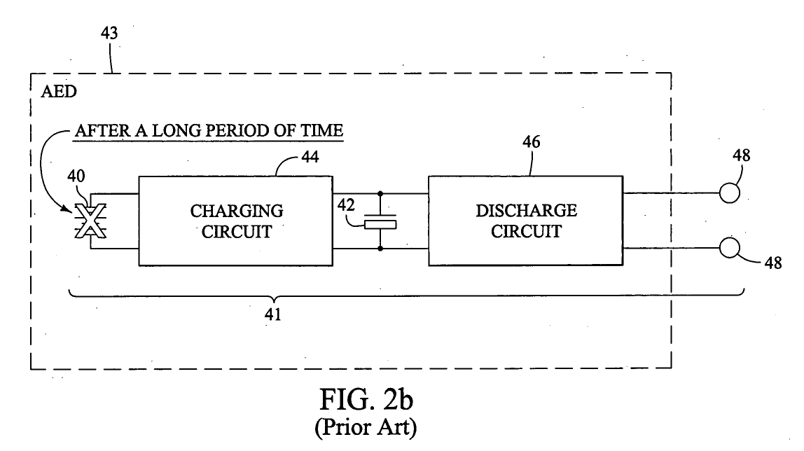

FIG. 3a is a perspective view of an embodiment for a portable defibrillator in accordance with the present invention;

FIG. 3b is a perspective view of an alternate embodiment for a portable defibrillator in accordance with the present invention;

FIG. 4a is a schematic of defibrillation and charging circuitry in accordance with an embodiment of the present invention;

FIG. 4b is a schematic of defibrillation and charging circuitry in accordance with an alternate embodiment of the present invention;

FIG. 4c is a schematic of defibrillation and emergency charging circuitry in accordance with yet another embodiment of the present invention;

FIG. 5 is a detailed schematic of defibrillation and line power charging circuitry in accordance with an embodiment of the present invention;

FIG. 6 is a detailed schematic of defibrillation and line power charging circuitry in accordance with an alternate embodiment of the present invention;

FIG. 7 is a detailed schematic of defibrillation and emergency charging circuitry in accordance with an embodiment of the present invention.

### DETAILED DESCRIPTION

The following detailed description is to be read with reference to the figures, in which like elements in different figures have like reference numerals. The figures, which are not necessarily to scale, depict selected embodiments and are not intended to limit the scope of the invention. Skilled artisans will recognize that the examples provided herein have many useful alternatives that fall within the scope of the invention.

The present invention is not limited to automatic external defibrillators and may be employed in many various types of electronic and mechanical devices for treating patient medical conditions such as external defibrillators. For purposes of illustration only, however, the present invention is below described in the context of automatic external defibrillators.

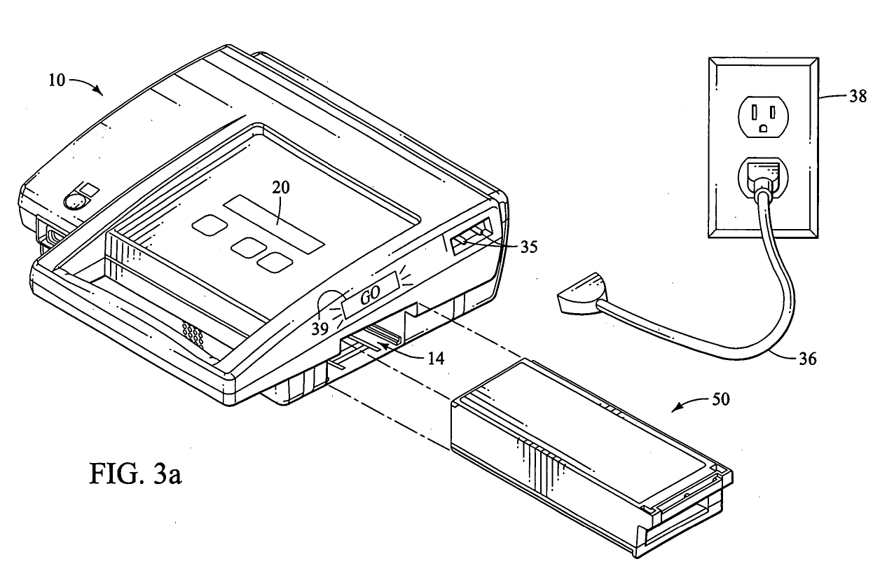

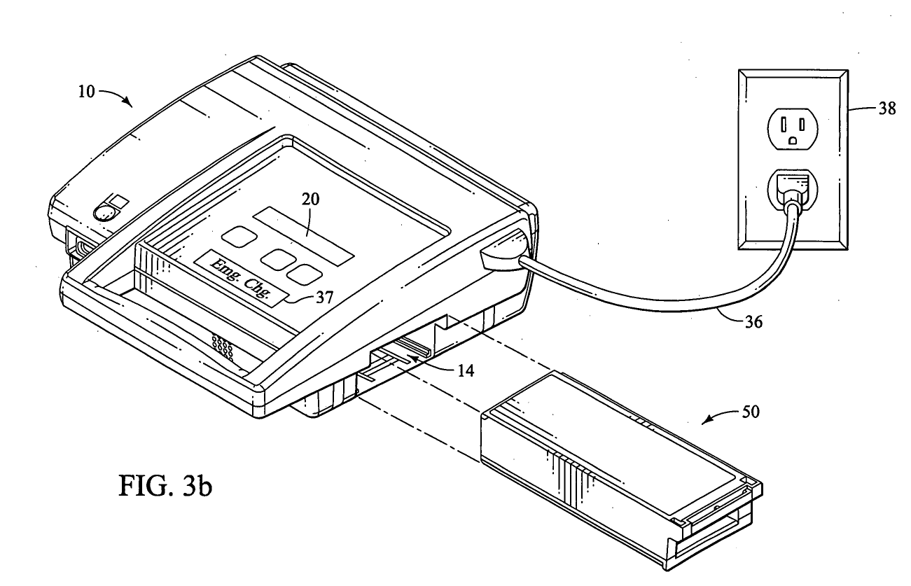

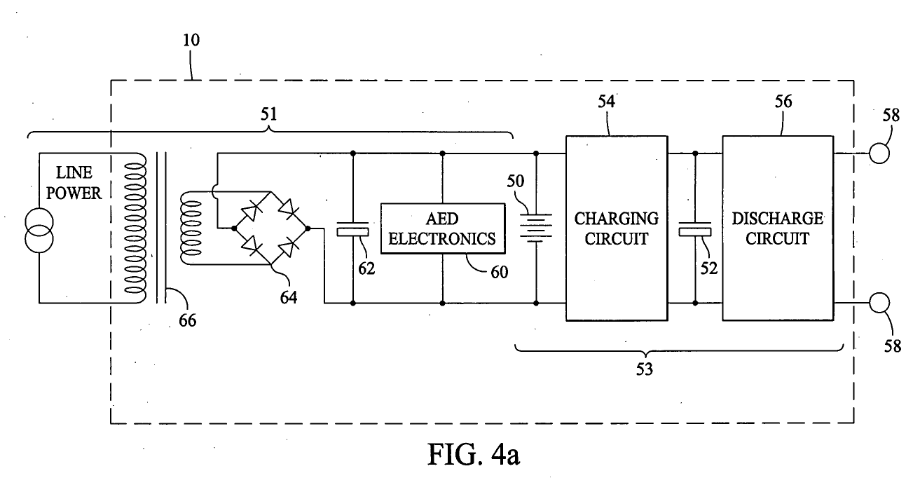

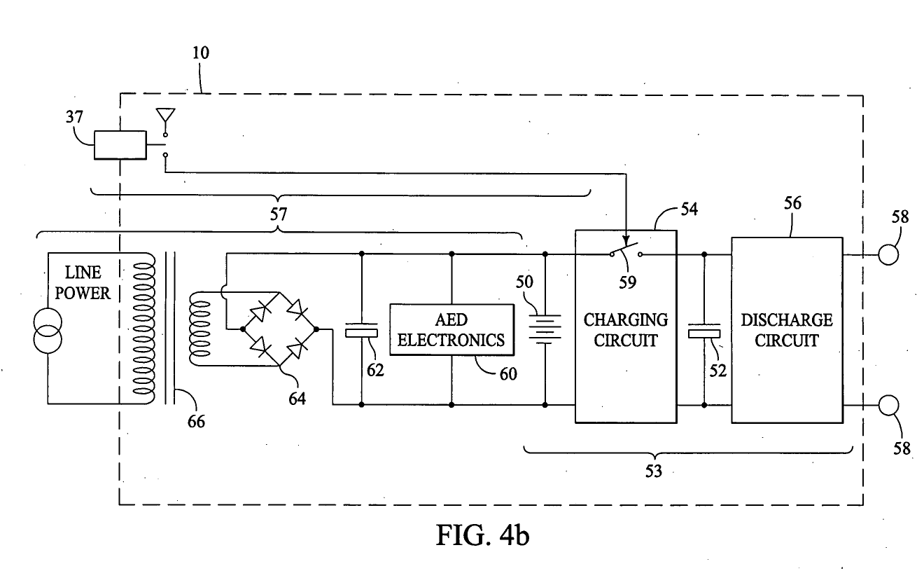

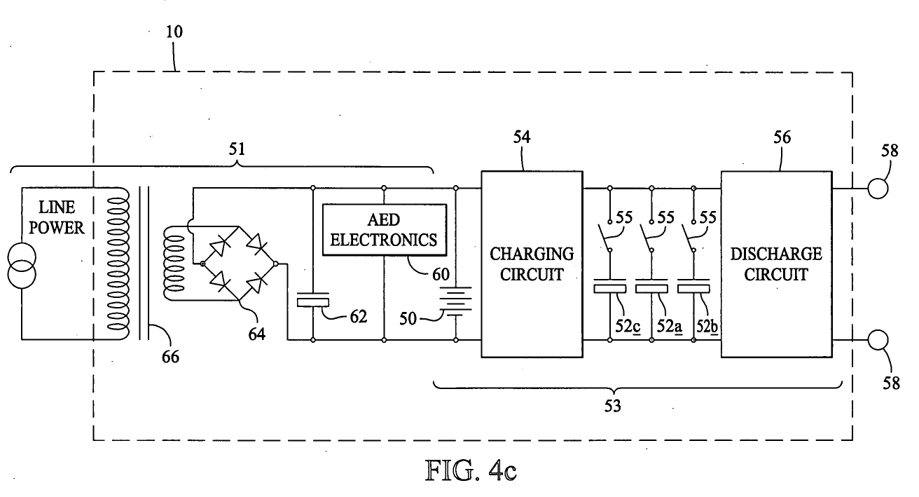

With reference to FIG. 3a and FIG. 4a, a perspective view of an embodiment for a portable defibrillator in accordance with the present invention is shown. AED 10 is capable of administering defibrillation therapy to a patient even if the AED's battery is depleted beyond the capacity to deliver defibrillation therapy. AED 10 includes one or more capacitors 52 that can generate one or more pulses to defibrillate the heart of a patient. The pulses may be delivered to the patient via two electrodes 58, which may be hand-held electrode paddles or adhesive electrode pads placed externally on the skin of the patient.

Electrodes 58 may be packaged in a sealed pouch, such as an airtight foil bag, which protects electrodes 58 from the environment. Electrodes 58 may include substances that may degrade or dry out when exposed to air. For example, electrodes 58 may include a hydrogel layer that hydrates the patient's skin, forms an interface with the patient, promotes adhesion of electrodes 58 to the skin and reduces the risk of burns. Electrodes 58 may be stored in a pouch to prevent the hydrogel from drying out and losing its desirable properties. The pouch may be stowed inside AED 10 or inside a cabinet.

An operator using AED 10 typically opens the pouch, retrieves electrodes 58, and places electrodes 58 in the correct position on the patient's chest. In some models of AED 10, the operator may also couple electrodes 58 to AED 10 by plugging an electrical connector into a receptacle on AED 10.

Electrodes 58 of the kind described above are intended for use on one occasion. Following use, electrodes 58 are discarded, and AED 10 may be supplied with a fresh pouch. Even if electrodes 58 are not used, however, electrodes 58 may have a shelf life. The pouch should be replaced when the shelf life expires.

AED 10 includes an internal power source that is typically an internal battery 50. Battery power is advantageous in many respects. First, in many situations, the patient may be far from an electrical outlet. In those situations, AED 10 may rely upon a battery 50 to supply the energy for the defibrillation pulses. Second, a power supply in the form of battery 50 makes AED 10 portable and useful in a wider variety of cardiac therapy situations. Battery 50 shown in FIG. 3a is a replaceable battery sliding into receiving slot 14. Further, battery 50 could be a rechargeable or non-rechargeable battery and still be in accordance with the spirit of the invention.

AED 10 also comprises one or more capacitors 52 and a charging circuit 54, such as a flyback charger. When a defibrillation pulse is needed, charging circuit 54 transfers energy from battery 50 to capacitor 52. When the energy stored in capacitor 52 reaches a desired level, AED 10 is ready to deliver at least one defibrillation pulse. In one embodiment, capacitor 52 can store enough voltage for two or more successive shocks. The therapy may be delivered automatically or manually.

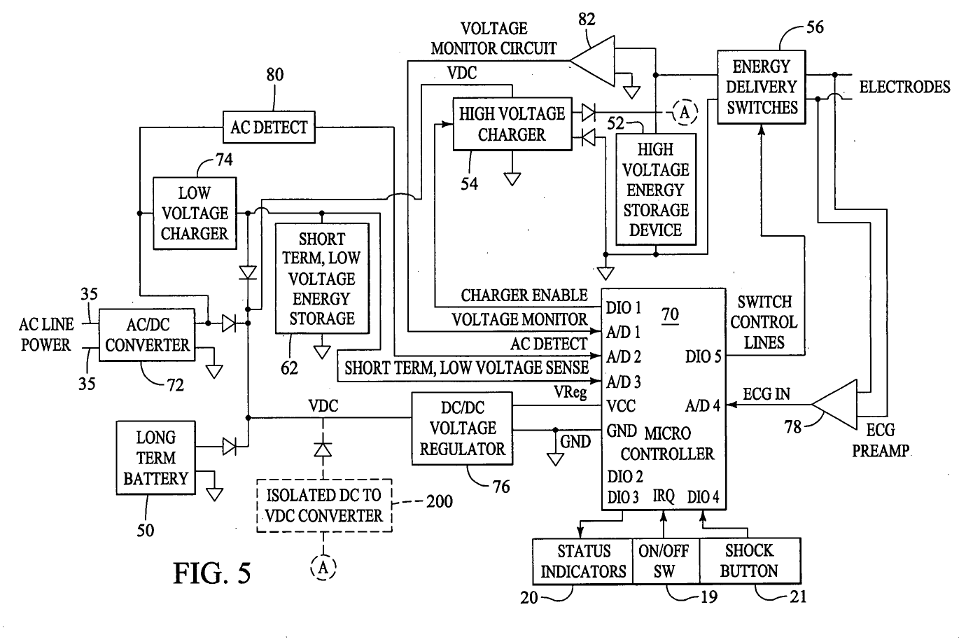

AED 10 may further include a microcontroller 70 (FIG. 5) as part of the AED's support electronics 60 that control various functions of AED 10 in support of delivering defibrillation pulses. Microcontroller 70 may govern charging of capacitor 52, for example, or may evaluate heart rhythms of the patient sensed via electrodes 58, or may deliver the defibrillation pulses automatically. Microcontroller 70 may further execute a routine performing a self-diagnostic test of AED 10 and acquire status information as a function of performing the self-diagnostic routine. Microcontroller 70 may further acquire ECG data collected during a use of AED 10 on a patient and/or scene audio information recorded during use on a patient.

Status information pertains to the operating status of AED 10 and its attendant components. Status information may include, for example, data indicative of AED 10 being in good working order. Status information may also include data indicative of a fault or potential problem with AED 10, such as data indicative of a failed or damaged component. Data indicating battery 50 is low, or battery 50 is failing to hold a charge, are additional examples of AED status information. Status information may also include data indicating electrodes 58 or other components are nearing the end of their shelf life, ECG data collected during use of AED 10 on a patient, and scene audio information recorded during use on a patient.

AED 10 may include one or more output elements 20 that convey status information to a person. As shown in FIG. 3a, output elements 20 include visual annunciators, such as light-emitting diodes (LEDs) that illuminate or darken to convey status information. Output elements 20 may, for example, indicate whether AED 10 is in good working order, whether battery 50 is ready, or whether AED 10 needs service. Output elements 20 may include other or additional annunciators, such as a liquid crystal display (LCD), a cathode ray tube (CRT) display, a strobe, or a speaker that is capable of delivering an audible signal or a spoken message.

The present invention overcomes the problems associated with the prior art by quickly and directly charging capacitor 52 from line power in the event of a cardiac emergency and battery 50 cannot provide enough energy for a defibrillation pulse. In accordance with the present embodiment, a power cord 36 electrically connects AED 10 with line power generally located at outlet 38. Power cord 36 plugs into power terminals 35 which receive line power from cord 36. Power terminals 35 then route the line power to emergency charging circuit 51 to charge capacitor 52. Typically the line power provides 110 V at 60 Hz. However, it is fully contemplated capacitor 52 could be charged from any line power source such as 220 V line power, an external 12 V battery, or any other power source of the like.

In the event of a cardiac emergency, the user will generally go to AED 10, pick it up, carry it to the victim, apply electrodes 58, turn AED 10 on, let AED 10 monitor the patient, and apply the proper therapy. If battery 50 is depleted to the point where it cannot provide enough energy for a defibrillation pulse, then the user can take AED 10 to the nearest outlet 38 and connect power cord 36 with power terminals 35 and outlet 38. Once plugged into the line power, capacitor 52 is able to charge generally within 10 or 15 seconds. This is very important since it is generally known the quicker a defibrillation pulse is administered the better odds the victim has of surviving a cardiac event. Once capacitor 52 is fully charged a light 39 illuminates indicating to the user capacitor 52 is fully charged and able to provide cardiac therapy. The user then unplugs AED 10 from power cord 36, carries AED 10 to the victim, applies the electrodes 58, turns AED 10 on, AED 10 monitors the patient, and applies the proper therapy. It is contemplated capacitor 52 does not need to be fully charged and can be charged to a sufficient voltage able to provide an adequate cardiac therapy.

With reference to FIG. 3b, another embodiment for a portable defibrillator in accordance with the present invention is shown. An emergency charging button 37 is provided to allow the user control over the emergency charging process. When emergency charging is needed, the user presses button 37 to begin charging capacitor 52 directly from line power. When capacitor 52 is finished charging, button 37 is reset and AED 10 is ready to provide cardiac therapy. Once button 37 is depressed line power from outlet 38 would be routed to emergency charging circuitry 51 which will be described in more detail below. The operation of the present invention is discussed further with reference to the Figures below. It is further contemplated emergency button 37 could break a battery charging circuit. For example, AED 10 could remain plugged into outlet 38 where the line power would charge battery 50. If a cardiac event occurred, and for some reason battery 50 was unavailable, then the user would push button 37 and AED would then stop recharging battery 50 and instead directly charge capacitor 52 from line power. It is further contemplated the line power would charge battery 50 after charging capacitor 52.

With reference to FIG. 4a, a schematic of defibrillation circuitry in accordance with an embodiment of the present invention is shown. In standard operation, regular defibrillation circuitry 53 comprises a battery 50, which provides a defibrillator charge to capacitor 52 through capacitor charging circuit 54. Capacitor 52 then provides a defibrillation pulse to a patient through discharge circuit 56 and electrodes 58. However, in the event of an emergency and battery 50 being substantially depleted, line power circuit 51 is able to charge capacitor 52 with sufficient energy to provide cardiac therapy. In operation during an emergency, when power cord 36 is plugged into outlet 38 and AED 10, line power is routed to transformer 66. Transformer 66 then steps down the line power to a more usable voltage for AED 10. This voltage is typically 10-12 V. The voltage then travels through a full wave rectifier 64 to provide 10-12 VDC. This voltage is then used to charge capacitor 52 via charging circuit 54 and super capacitor 62. It is fully contemplated transformer 66 and rectifier 64 could be located external to AED 10. For example, transformer 66 and rectifier 64 could be located in cord 36 such as a direct current adapter. In this example, the line power would be converted to DC before entering AED 10 and emergency circuitry 51. It is further contemplated the frequency of the AC line power could be increased via a frequency multiplier in order to reduce the necessary size of transformer 66 and thus the size of AED 10. That is, the size of the necessary windings on transformer 66 is reduced when stepping down higher frequency line power.

Super capacitor 62 is one or more low voltage high capacitance capacitors that charge quickly. Typically, capacitor 62 has a capacitance in the hundreds of farads. Capacitor 62 is used to power defibrillator electronics 60 such as microcontroller 70 and output elements 20 during emergency operation of AED 10. It is contemplated microcontroller 70 could be powered from line power during the emergency charging of capacitors 62 and 52. Super capacitors typically charge to approximately 2.5 volts. Therefore, three or more super capacitors 62 are typically stacked in series to provide enough voltage to run defibrillator electronics 60. Capacitors 62 can also hold their charge for several hours. As an alternative, a low voltage, fast charging emergency battery could be used in the place of capacitor 62. Further, it is contemplated if battery 50 had a charge remaining, which is not large enough to provide a defibrillation pulse, but large enough to power microcontroller 70, then battery 50 could provide an alternate low power source instead of capacitor 62. Regardless of which device powers defibrillator electronics 60, it is contemplated the emergency battery, capacitor 62, or battery 50 will provide enough power sufficient to operate the patient diagnostic circuitry at least until one defibrillation shock is delivered.

Defibrillation capacitor 52 is capable of charging to a much higher voltage than super capacitor 62, typically in the range of 1700-2100 volts. As stated capacitor 52 is utilized to provide a defibrillation pulse to a patient as is known in the art. Typically, AED 10 is not plugged into outlet 38 unless there is an emergency where it is necessary to charge capacitor 52 quickly. It is damaging to capacitor 52 to leave it charged for long periods. Preferably capacitor 52 and capacitor 62 are charged at the same time and low storage capacitor 62 can support running diagnostics on a single charge for multiple defibrillation pulses.

With reference to FIG. 4b, a schematic of defibrillation circuitry in accordance with an alternate embodiment of the present invention is shown. Although circuitry 57 of FIG. 4b is similar to circuitry 51 in FIG. 4a, circuitry 57 provides an emergency charging switch 37, which allows capacitor 52 to selectively receive line power when in a battery emergency mode. Thus, the charging circuit's selective receipt of line power in FIG. 4b is contrasted from the embodiment of FIG. 4a, where capacitor 52 is charged automatically after AED 10 is connected to line power. When switch 37 is pressed an enable signal is sent which allows capacitor 52 to be charged from line power. In the present embodiment, an enable signal is sent to close switch 59 from emergency switch 37, which allows line power to charge capacitor 52. When capacitor 52 is fully charged, switch 59 is opened until AED 10 is needed for emergency operation. It is contemplated that switch 59 may be selectively closed by any combination of factors, including switch 37, receipt of a low battery signal, etc. It is contemplated switch 59 could be closed in many number of ways. For example, opening a cover of AED 10 could close switch 59. Further, it is contemplated activation of switch 59 could be activated remotely. For example, it could be activated (via a wired or wireless link) by the act of deployment of AED 10. In a commercial or public access location this could be done by emergency dispatch or local security triggering the charge activation upon receipt of an emergency call and location determination of the nearest AED. The responder would be instructed to proceed to the AEDs location and retrieve the now fully charged AED 10. The opening of a storage cabinet or enclosure could activate switch 59 where AED 10 is located. Switch 59 could be activated by a proximity switch or sensor located on or near AED 10. Thus when there is a demonstrated intent to use AED 10 the charging is activated.

With reference to FIG. 4c, a schematic of defibrillation circuitry in accordance with another embodiment of the present invention is shown. In contrast to the embodiment of FIG. 4b, this embodiment has three defibrillation capacitors 52a, 52b, and 52c, which are all charged in parallel from charging circuit 54. Switches 55 isolate each capacitor 52a, 52b, and 52c from each other during discharge of any one of capacitors 52a, 52b, and 52c. The present embodiment allows the user to charge not one, but a plurality of capacitors from the line power. Each capacitor 52a, 52b, and 52c is then isolated creating three defibrillation pulses stored. This embodiment has an advantage since a percentage of cardiac arrest patients require two or more defibrillation pulses.

With reference to FIGS. 5, 6, and 7, a detailed schematic of defibrillation and emergency charging circuitry in accordance with three different embodiments of the present invention is shown. In all three embodiments, microcontroller 70 is in a state of consuming less power than in its regular operation. This can be a sleep mode or other mode as known by those skilled in the art. Further, for any of the implementations, AED 10 does not have to appear to be operational when connected to outlet 38 (e.g., AED 10 is off). Even in the embodiment of FIG. 5 where microcontroller 70 controls charging capacitor 52, microcontroller 70 can either power up when connected to AC line power, or operate in a background mode requiring a user to press ON/OFF button 19 before operating normally. This embodiment has the advantage of being less confusing to a user by always requiring the user to press ON button 19. It also avoids requiring the user to turn on AED 10 to charge capacitor 52. It is preferable for all three embodiments capacitor 52 be charged after AED 10 is connected to line power, typically before AED 10 is turned on.

With reference to FIG. 5, the user is alerted to this via output elements 20 when battery 50 is unable to provide a defibrillation pulse. The user may then plug AED 10 into outlet 38 through cord 36 to charge capacitor 52 via line power. In the embodiment of FIG. 5, microcontroller 70 is activated from a low power consumption mode after AED 10 is connected to line power. However, if battery 50 is totally depleted, microcontroller 70 is powered up when it receives the AC detect signal and the low voltage sense signal. AC line power then travels through power terminals 35 and into AC/DC converter 72 that houses transformer 66 and rectifier 64 (discussed above). The output of converter 72 is a DC voltage at approximately 12 volts. This DC voltage is received by AC detect circuit 80, which detects the AC ripple in the DC voltage signal and then outputs an AC detect signal to microcontroller 70. The DC voltage signal is also received by regulator 76, which takes the DC signal and cleans up the AC ripple and outputs a 5 volt voltage regulated signal which powers microcontroller 70. It is contemplated converter 72 further houses a frequency multiplier circuit to increases the frequency of the line power. This increase in frequency assists in reducing the size of transformer 66 and thus reduces the size of AED 10. In one embodiment the frequency multiplier circuit would increase the frequency of the line power before the power is supplied to transformer 66.

When microcontroller 70 receives the AC detect signal, microcontroller 70 is activated from a low power mode. However, if battery 50 is totally depleted, microcontroller 70 is powered up when it receives the AC detect signal and the low voltage sense signal. Thus microcontroller 70 will have voltage in which to operate. Microcontroller 70 then sends a charger enable signal to charging circuit 54 instructing it to begin charging capacitor 52. Thus, microcontroller 70 then controls the charging of capacitor 52. Using the stepped down and rectified line power voltage from converter 72, charging circuit 54 then begins to charge capacitor 52. The charging continues until sensor 82 detects when capacitor 52 is charged to a preset value and outputs a voltage monitor signal to microcontroller 70. During charging of capacitor 52, low voltage charger 74 receives DC voltage from converter 72. Charger 74 then reduces the DC voltage signal from charger 72 and outputs a voltage that charges capacitor 62 and provides a low voltage sense signal to microcontroller 70. This low voltage signal informs microcontroller 70 capacitors 62 are charging and microcontroller 70 will have a supply of voltage in which to operate after AED 10 is unplugged from outlet 38. When capacitor 52 is fully charged the user is informed AED 10 is ready to administer a defibrillation pulse by indicator 39 and any of output elements 20, such as an LED or an audible alarm. The user may then unplug AED 10, take AED 10 to the patient, place electrodes 58 on the patient, and turn on AED 10. The operations circuitry 54, 56, 60 that provide monitoring and control functions for AED 10 are then powered by the charge from capacitors 62. AED 10 then monitors ECG signals from the patient. The ECG signal is detected from the patient through ECG preamp 78. This ECG signal is then sent to microcontroller 70 where the ECG data is processed and it is determined whether a defibrillation pulse should be administered. If a defibrillation pulse is appropriate, microcontroller 70 sends a signal to discharge circuit 56, which then discharges the energy stored in capacitor 52 into the patient through electrodes 58. The discharge process may occur automatically or semi-automatically where a manual pulse button 21 allows the user to apply the pulse if indicated.

Alternatively, as shown by the dotted lines, capacitor 52 could provide the energy to operations circuitry 54 to monitor and control AED 10 operation while also providing defibrillation pulses to a patient. In this embodiment, energy would be transferred to node A and then to Isolated DC to VDC Converter 200, which drops the high voltage from capacitor 52 down to battery voltage levels. The down converted voltage from capacitor 52 through isolated DC to VDC Converter 200 would pass though voltage regulator 76 to become regulated DC. The regulated DC would then be utilized by the operations circuitry 54 to monitor and control AED 10.

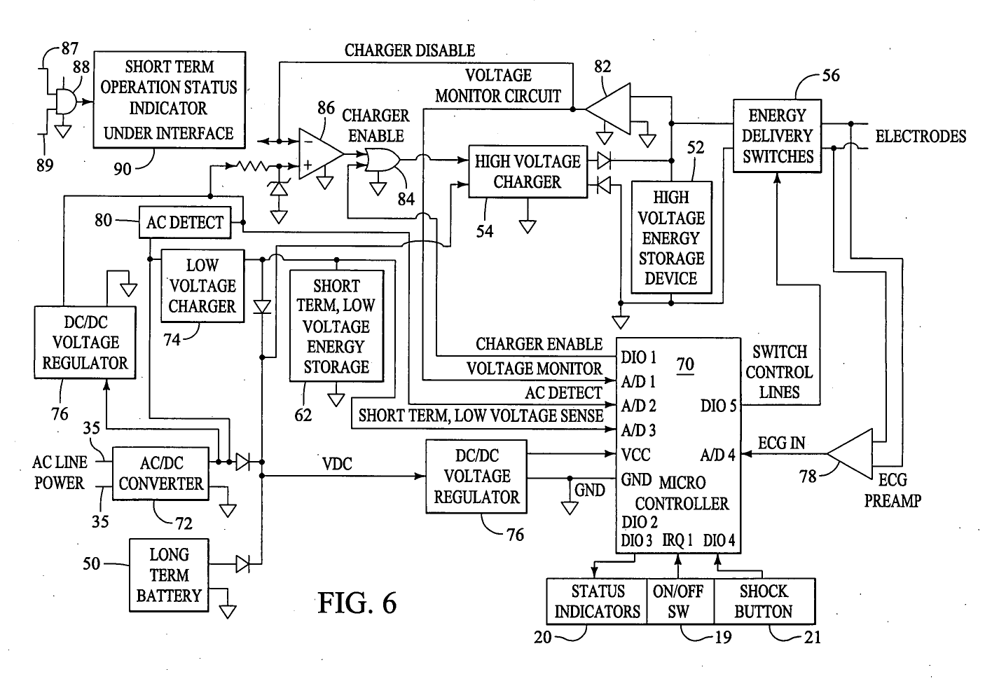

With reference to FIG. 6, the circuitry is similar to the embodiment of FIG. 5. However, in the embodiment of FIG. 6 microcontroller 70 is not needed to charge capacitor 52. Nevertheless microcontroller 70 is utilized to perform monitoring and control functions. That is, microcontroller 70 analyzes the patient's ECG signals and then delivers a defibrillation pulse if indicated. When line power is connected to AED 10, AC detect circuit 80 produces a voltage on regulation switch 86. Regulation switch 86 will then output a voltage to OR gate 84. OR gate 84 then outputs a voltage to charging circuit 54, causing charging circuit 54 to begin charging capacitor 52 with voltage received from converter 72. Sensor 82 then detects when capacitor 52 is fully charged and outputs a voltage monitor signal to regulating switch 86 and microcontroller 70. Receiving both an AC detect signal and a full charge signal, switch 86 then drops its output to OR gate 84 to a low voltage or zero voltage. Upon receipt of the voltage monitor signal from sensor 82, microcontroller 70 also drops its output (charger enable signal) to OR gate 84 to a low state. Therefore, OR gate 84 outputs a low or zero voltage, which stops circuit 54 from charging capacitor 52.

In addition, microcontroller, while not being necessary to charge capacitor 52, can also initiate the charging of capacitor 52. When microcontroller 70 receives the AC detect signal from AC detect circuit 80, microcontroller 70 is activated from a low power mode. Microcontroller 70 then sends a charger enable signal to OR gate 84. OR gate 84 then outputs a voltage to charging circuit 54, which then begins to charge capacitor 52 with voltage received from converter 72. Sensor 82 then detects when capacitor 52 is fully charged and outputs a voltage monitor signal to regulating switch 86 and microcontroller 70. Switch 86 then outputs a low voltage or zero voltage to OR gate 84 and microcontroller 70 changes the charger enable signal to a low state. Therefore, OR gate 84 outputs a low or zero voltage, which stops charging circuit 54 from charging capacitor 52.

In this embodiment, the user is informed of AED 10 being ready to provide an emergency defibrillator pulse. When capacitor 52 is fully charged, regulation switch 86 sends a signal to line 87, which provides a high state at AND gate 88. When capacitor 62 is fully charged (indicating an emergency battery condition), microcontroller 70 sends a low voltage sense signal to line 89, which provide a high state at AND gate 88. With all of the inputs to AND gate 88 at a high state, gate 88 outputs a signal which illuminates short term operation status light 90 and informs the user AED 10 is ready for use. Advantageously in this embodiment, microcontroller 70 function is not necessary to charge capacitor 52. Therefore, the operational mode (e.g., position of on/off switch 19) of microcontroller 70 is of no consequence to the charging of capacitor 52.

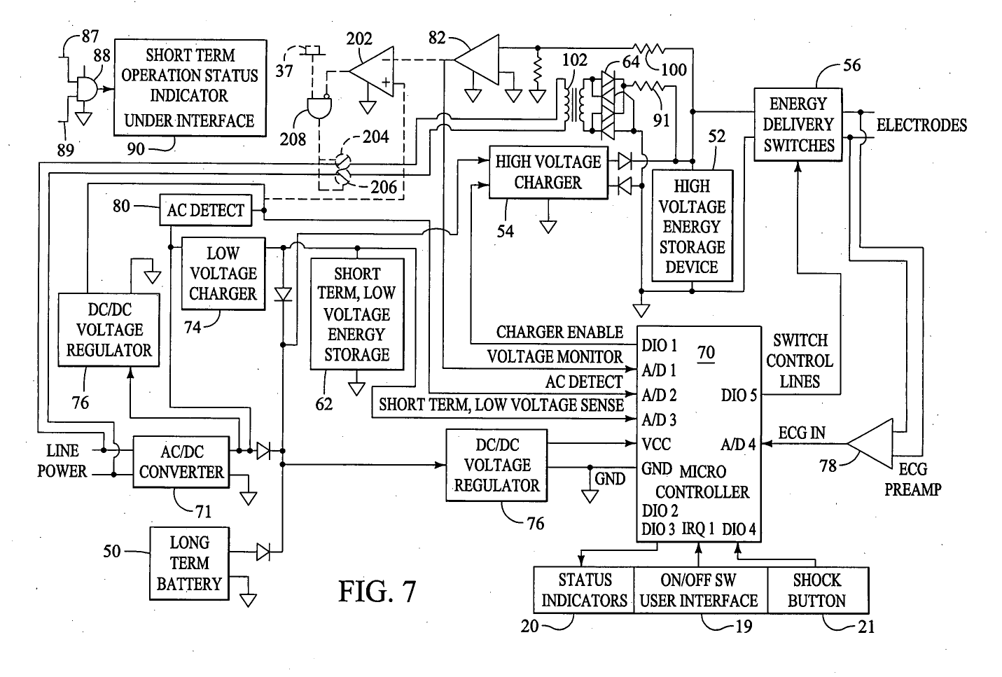

With reference to FIG. 7, microcontroller 70 and charging circuit 54 are bypassed completely when AC line power charges capacitor 52. The charging circuit for emergency charging is distinct from the main charging circuit used when the battery is sourcing charging power. When line power is connected to AED 10, it travels directly to transformer 102, which steps up the voltage to approximately 1,700-2,000 volts. Rectifier 64 then converts the line voltage to DC and outputs the voltage to capacitor 52. It is of note that resistive load 91 acts as an input impedance to attenuate the in-rush current to the capacitor. Sensor 82 along with voltage divider 100 detects when capacitor 52 is fully charged and sends a voltage monitor signal to microcontroller 70. When capacitor 52 is fully charged, sensor 82 sends a signal to line 87, which provides a high voltage at AND gate 88. When capacitor 62 is fully charged, microcontroller 70 sends a signal to line 89, which provide a high voltage at AND gate 88. With all of the inputs to AND gate 88 at a high state, gate 88 outputs a signal which illuminates short term operation status light 90 and informs the user AED 10 is ready for use.

An advantage of this embodiment is microcontroller 70 is not necessary in order to charge capacitor 52. However microcontroller 70 can assist in the charging of capacitor 52. When microcontroller 70 receives the AC detect signal from AC detect circuit 80 then microcontroller 70 is activated from a low power mode. Microcontroller 70 then sends a charger enable signal to charging circuit 54. Charging circuit 54 then begins to charge capacitor 52 with voltage received from converter 71, which operates similarly to converter 72. Now capacitor 52 can be charged from two sources thus reducing the amount of time necessary to charge capacitor 52. Further, the voltage from converter 71 is used to charge capacitor 62. Therefore, in this embodiment microcontroller 70 is not needed to charge capacitor 52 and capacitor 52 and 62 have separate charging circuits to ensure each is charged as quickly as possible.

In another embodiment of FIG. 7, a user button 37 could close switches 204 and 206 in-line with AC power. Switches 204 and 206 are normally kept open (i.e., latches the in-line power switches open). Preferably, button 37 is a latch armed when AC line power is connected. Therefore, user button 37 is activated only when AED 10 is plugged into line power. When AED 10 is plugged into line power, button 37 is illuminated indicating to the user AED 10 is ready to directly charge capacitor 52. The user then pushes button 37, which sends a high state to AND gate 208. If capacitor 52 is not charged, regulations switch 202 will output a low state which is inverted to AND gate 208. AND gate 208 then outputs a high state, which latches the in-line power switches 204 and 206 closed so charging of capacitor 52 occurs rapidly. When capacitor 52 is fully charged sensor 82 detects this and sends a high state signal, which is inverted low, to regulation switch 202. AND gate 208 then outputs a low signal which opens switches 204 and 206 and resets button 37. Switches 204 and 206 will then remain closed until AED 10 is plugged into line power again. In an alternate embodiment button 37 is only rearmed or reset if AC line power is disconnected.

In this embodiment, the user also has the option to use button 37 as an interrupt switch. For example, if AED 10 has been plugged in and button 37 was depressed to begin capacitor 52 charging, the user could press button 37 once more to stop capacitor 52 from charging. In effect, when the user presses button 37 a second time, a low state signal is sent to AND gate 208, which in combination with a low state inverted high from regulation switch 202 causes AND gate 208 to output a low state signal and open switches 204 and 206.

One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A portable defibrillator comprising:
a capacitor adapted to receive electrical charge to deliver a defibrillation charge from a main battery via a regular current path;
power terminals to receive line power; and
an emergency charging circuit to charge the capacitor from the power terminals via an emergency current path distinct from the regular current path.

2. The defibrillator of claim 1, in which the line power is provided as alternating current, and the emergency charging circuit further includes a transformer.

3. The defibrillator of claim 1, in which the line power is provided as alternating current, and the emergency charging circuit includes a line power rectifier.

4. The defibrillator of claim 3, in which the emergency charging circuit further includes an input impedance to attenuate in-rush current to the capacitor.

5. The defibrillator of claim 1, further comprising a sensing circuit to sense when the capacitor has been charged up to a preset voltage.

6. The defibrillator of claim 5, further comprising a regulation switch to interrupt charging the capacitor responsive to an input from the sensing circuit.

7. The defibrillator of claim 5, in which the sensing circuit includes a resistive voltage divider.

8. The defibrillator of claim 5, further comprising an alarm device to issue an alarm responsive to an input from the sensing circuit.

9. The defibrillator of claim 8, in which the issued alarm is visual.

10. The defibrillator of claim 8, in which the issued alarm is auditory.

11. The defibrillator of claim 8, in which the issued alarm is a status indicator.

12. The defibrillator of claim 1, further comprising an emergency switch to activate the emergency charging circuit.

13. The defibrillator of claim 1, further comprising an emergency battery connected to be charged from the power terminals.

14. The defibrillator of claim 13, further comprising patient diagnostic circuitry operable from the emergency battery.

15. The defibrillator of claim 13, wherein the line power charges the emergency battery to a level sufficient to operate the patient diagnostic circuitry at least until one defibrillation shock is delivered.

16. The defibrillator of claim 1, further comprising patient diagnostic circuitry operable from the capacitor.

17. The defibrillator of claim 1, further comprising a short term, low voltage energy storage module.

18. The defibrillator of claim 17, in which the storage module gets charged along with the capacitor and which can support running diagnostics circuits on a single charge for a single shock.

19. The defibrillator of claim 1, wherein the emergency current path includes at least a portion of the regular current path.

20. The defibrillator of claim 1, further comprising: a microcontroller, and in which the received line power powers the microcontroller.

21. The defibrillator of claim 1, further comprising: a microcontroller, and in which the received line power does not power the microcontroller.

22. The defibrillator of claim 1, wherein the portable defibrillator is a fully automated defibrillator.

23. The defibrillator of claim 1, wherein the portable defibrillator is a semi automatic defibrillator.

24. A portable defibrillator comprising:
a capacitor adapted to receive electrical charge to deliver a defibrillation charge;
power terminals to receive line power; and
a charging circuit to charge the capacitor from line power after the power terminals receive line power.

25. The defibrillator of claim 24, in which the line power is provided to the power input terminals as rectified by a direct current adapter.

26. The defibrillator of claim 24, wherein the charging circuit automatically charges the capacitor after the power terminals receive line power.

27. The defibrillator of claim 24, wherein the charging circuit selectively charges the capacitor after the power terminals receive line power.

28. The defibrillator of claim 27, further comprising an emergency switch that selects whether the charging circuit charges the capacitor.

29. The defibrillator of claim 24, wherein the charging circuit begins charging the capacitor from the power terminals when the power terminals receive line power.

30. The defibrillator of claim 24, wherein the charging circuit charges the capacitor to a level sufficient for one shock.

31. The defibrillator of claim 30, wherein the charging circuit charges the capacitor to a level sufficient for one defibrillation shock within about 10 to 15 seconds after the power terminals receive line power.

32. The defibrillator of claim 24, further comprising a main battery adapted to charge the capacitor.

33. The defibrillator of claim 32, wherein a main charging circuit charges the capacitor from battery power.

34. The defibrillator of claim 33, wherein the charging circuit is an emergency charging circuit, the main charging circuit being distinct from the emergency charging circuit.

35. The defibrillator of claim 33, wherein the main charging circuit is the charging circuit.

36. The defibrillator of claim 32, wherein the line power charges the battery after charging the capacitor.

37. The defibrillator of claim 24, further comprising a line power detection sensor that signals the charging circuit to begin charging the capacitor when the sensor detects line power.

38. The defibrillator of claim 37, further comprising control circuitry, the control circuitry providing a charge enable signal to initiate the charging of the capacitor via the charging circuit.

39. The defibrillator of claim 38, wherein the charging circuit initiates charging of the capacitor after receiving signaling from the sensor or after the control circuitry provides the charge enable signal.

40. The defibrillator of claim 38, wherein the control circuitry provides the charge enable signal once the sensor detects line power.

41. The defibrillator of claim 24, further comprising a user on/off switch to activate defibrillator operation, the charging circuit charging the capacitor independent of the user on/off switch.

42. The defibrillator of claim 24, further comprising a sensing circuit to sense when the capacitor has been charged up to a preset voltage.

43. The defibrillator of claim 42, further comprising a regulation switch to interrupt charging the capacitor responsive to an input from the sensing circuit.

44. The defibrillator of claim 24, further comprising a step down transformer that reduces the voltage level of the line power before supplying it to the charging circuit.

45. The defibrillator of claim 44, further comprising a frequency multiplier circuit that increases the frequency of the line power before it is supplied to the step down transformer.

46. The defibrillator of claim 24, wherein the portable defibrillator is a fully automated defibrillator.

47. The defibrillator of claim 24, wherein the portable defibrillator is a semi automatic defibrillator.

48. The defibrillator of claim 24, wherein the capacitor can store enough charge for at least two successive defibrillation shocks.

49. A defibrillator comprising:
a first capacitor suited to deliver a first defibrillation shock to a patient; and
a second capacitor adapted to deliver a second defibrillation shock.

50. The defibrillator of claim 49, further comprising:
a main battery for charging the first capacitor; and
power terminals to receive line power and to charge the second capacitor with the received line power.

51. The defibrillator of claim 50, in which the first capacitor may also be charged through the received line power.

52. The defibrillator of claim 49, further comprising: a microcontroller, and in which the received line power powers the microcontroller.

53. The defibrillator of claim 49, further comprising: a microcontroller, and in which the received line power does not power the microcontroller.

54. A portable defibrillator comprising:
a capacitor adapted to receive electrical charge to deliver a defibrillation charge;
power terminals to receive line power;
operations circuitry adapted to monitor and control defibrillator operation and to be powered by a main battery;
a short term, low voltage energy storage module capable of providing emergency power to the operations circuitry when charged; and
a low voltage charging circuit to charge the storage module from the line power after the power terminals receive line power.

55. The defibrillator of claim 54, further comprising a capacitor adapted to receive electrical charge to deliver a defibrillation charge, and a capacitor charging circuit to charge the capacitor after the power terminals receive line power.

56. The defibrillator of claim 55, wherein the storage module can power the operations circuitry on a single charge for a single shock.

57. The defibrillator of claim 54, wherein the storage module is a super capacitor.

58. The defibrillator of claim 57, wherein the super capacitor has a capacitance in the hundreds of Farads.

59. The defibrillator of claim 54, wherein the storage module is an emergency battery.

60. The defibrillator of claim 54, wherein the portable defibrillator is a fully automated defibrillator.

61. The defibrillator of claim 54, wherein the portable defibrillator is a semi automatic defibrillator.

62. The defibrillator of claim 54, wherein the capacitor can store enough charge for at least two successive defibrillation shocks.

63. A method comprising:
procuring for immediate use a defibrillator having a capacitor;
determining that a battery of the defibrillator is too depleted for charging through a regular current path the capacitor adequately for a defibrillation shock; and
connecting the defibrillator to a source of line power for emergency charging the capacitor through an emergency current path.

64. The method of claim 63, further comprising activating an emergency switch to enable the emergency charging.

65. The method of claim 63, in which activating the emergency switch is by opening a cover of the defibrillator.

66. The method of claim 63, further comprising waiting for an issued alarm to indicate the capacitor has been charged, and then using the defibrillator.

67. The method of claim 63, wherein the defibrillator has a second capacitor, and the emergency charging charges both capacitors.

68. The method of claim 67, further comprising delivering a first defibrillation shock from the charge on the capacitor, and delivering a second defibrillation shock from the charge on the second capacitor.

69. A method of charging a defibrillation capacitor in a portable defibrillator when the defibrillator contains insufficient power to charge the capacitor, comprising:
connecting the defibrillator to line power;
stepping up the voltage level of the line power;
rectifying the stepped up line power voltage;
supplying the rectified line power to the capacitor.

70. The method of claim 69, further comprising sensing when the capacitor has been charged up to a preset voltage.

71. The method of claim 70, further comprising interrupting the supply of rectified line power to the capacitor after the capacitor has been charged up to the preset voltage.

72. The method of claim 70, further comprising issuing an alarm after the capacitor has been charged up to the preset voltage.

73. The method of claim 69, further comprising manually activating a switch before supplying rectified line power to the capacitor.

74. The method of claim 69, wherein the defibrillator has a second capacitor, and the supply of rectified line power is supplied to charge both capacitors.

75. The method of claim 74, further comprising delivering a first defibrillation shock from the charge on the capacitor, and delivering a second defibrillation shock from the charge on the second capacitor.

76. The method of claim 69, further comprising attenuating the current level of the rectified line power before supplying the rectified line power to the capacitor.

77. A method of charging a defibrillation capacitor in a portable defibrillator when the defibrillator contains insufficient power to charge the capacitor, comprising:
connecting the defibrillator to line power;
stepping down the voltage level of the line power;
rectifying the stepped up line power voltage;
stepping up the voltage level of the rectified line power; and
supplying the rectified line power to the capacitor.
